(19) United States
(12) Patent Application Publication
(10) Pub. No.: US 2025/0382313 A1
(43) Pub. Date: Dec. 18, 2025

(54) BISPHOSPHITES WITH A 2-PHENYLPHENOL OUTER UNIT

(71) Applicant: Evonik Oxeno GmbH & Co. KG, Marl (DE)

(72) Inventors: Julia Büker, Bochum (DE); Robert Franke, Marl (DE); Dirk Fridag, Haltern am See (DE); Marius Bilke, Datteln (DE); Bastian Bruns, Bochum (DE); Vincent Bürk, Recklinghausen (DE); Peter Kucmierczyk, Herne (DE); Ana Markovic, Haltern am See (DE); Roland Peschke, Duisburg (DE); Maximilian Wessner, Dortmund (DE); Anna Chiara Sale, Düsseldorf (DE)

(73) Assignee: Evonik Oxeno GmbH & Co. KG, Marl (DE)

(21) Appl. No.: 19/236,626

(22) Filed: Jun. 12, 2025

(30) Foreign Application Priority Data
Jun. 14, 2024 (EP) 24182187.5

(51) Int. Cl. C07F 9/141; C07C 29/16

(52) U.S. Cl. CPC C07F 9/141; C07C 29/16; C07C 2523/36

(57) ABSTRACT

Bisphosphites with a 2-phenylphenol outer unit and the use thereof in hydroformylation.

The invention relates to bisphosphites with a 2-phenylphenol outer unit and to the use thereof in hydroformylation.

Phosphorus-containing compounds play a crucial role as ligands in a multitude of reactions, e.g. in hydrogenation, in hydrocyanation and also in hydroformylation.

WO 02/00670 A1 describes bisphosphite compounds, metal complexes thereof and use of the compounds and complexes in olefin hydroformylation. Disclosed here, inter alia, is the compound (Ila):

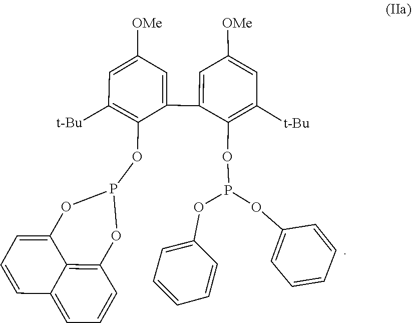 [SMILES: COc1cc(-c2cc(OC)cc(C(C)(C)C)c2OP2Oc3cccc4cccc(c34)O2)c(OP(Oc2ccccc2)Oc2ccccc2)c(C(C)(C)C)c1]

The technical object of the invention is to provide a compound with which a good n-selectivity can be achieved in the hydroformylation of olefins.

The object is achieved by a compound according to claim 1.

Compound of formula (I):

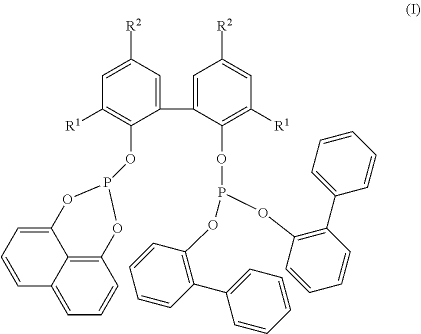

wherein R¹, R² are selected from: —H, —(C₁-C₆)-alkyl, —O—(C₁-C₆)-alkyl.

In one embodiment, R¹, R² are selected from: —(C₁-C₆)-alkyl, —O—(C₁-C₆)-alkyl.

In one embodiment, R¹ is —(C₁-C₆)-alkyl.

In one embodiment, R¹ is -^{t}Bu.

In one embodiment, R² is selected from: —(C₁-C₆)-alkyl, —O—(C₁-C₆)-alkyl.

In one embodiment, R² is -^{t}Bu or —O—CH₃.

In one embodiment, R² is -^{t}Bu.

In one embodiment, R² is —O—CH₃.

In one embodiment, the compound has the structure (1) or (2):

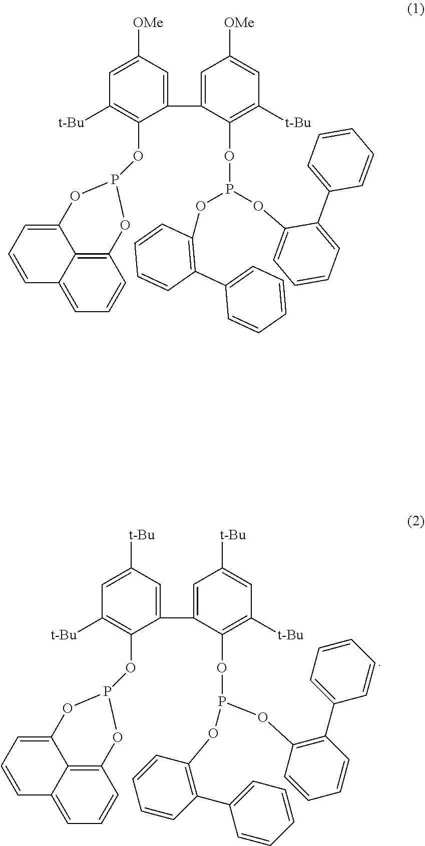 [SMILES: COc1cc(-c2cc(OC)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1 ; CC(C)(C)c1cc(-c2cc(C(C)(C)C)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

In one embodiment, the compound has the structure (1):

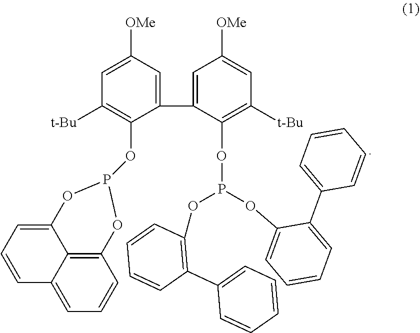 [SMILES: COc1cc(-c2cc(OC)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

In one embodiment, the compound has the structure (2):

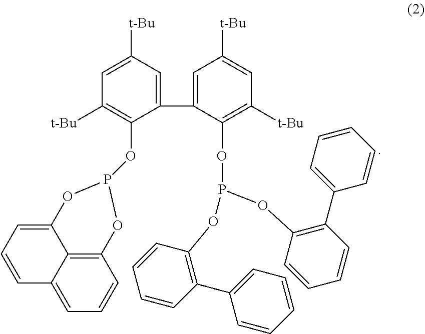 [SMILES: CC(C)(C)c1cc(-c2cc(C(C)(C)C)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

In addition to the compounds themselves, a process in which the compounds described above are used is also claimed.

Process comprising the process steps of:
a) initially charging an olefin;
b) adding a compound described above;
c) adding a substance comprising Rh;
d) feeding in H₂ and CO;
e) heating the reaction mixture from a) to d), with conversion of the olefin to an aldehyde.

In one variant of the process, the substance comprising Rh is selected from: Rh(acac)(CO)₂, Rh(acac)(cod) (Umicore, acac=acetylacetonate anion; cod=1,5-cyclooctadiene), Rh₄CO₁₂.

In one variant of the process, the substance comprising Rh is Rh(acac)(CO)₂.

The invention is to be elucidated in more detail hereinafter with reference to exemplary embodiments.

### Synthesis

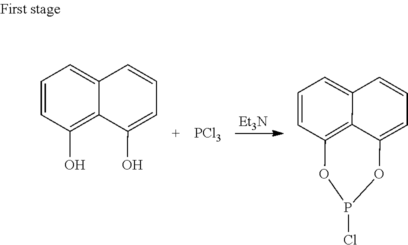

0.076 mol of naphthalene-1,8-diol was dried overnight at 50° C. by means of oil-pump vacuum. The following day, the Schlenk flask was flooded with argon and the naphthalene-1,8-diol was dissolved in 350 ml of dried toluene. In a Schlenk flask which had been repeatedly evacuated and filled with inert gas, 0.114 mol of phosphorus trichloride was dissolved in 120 ml of dried toluene. Subsequently, the naphthalene-1,8-diol solution was slowly and steadily added dropwise at −20° C. to the PCl₃ solution. Thereafter, 0.165 mol of triethylamine was slowly added dropwise at −20° C. to the solution under high stirring speed. The solution was brought to room temperature and stirring was continued overnight. The next day, the reaction mixture was filtered using a frit, the filter cake was washed twice with in each case 25 ml of toluene and the filtrate was concentrated at 40° C. by means of oil-pump vacuum.

Yield: 86%

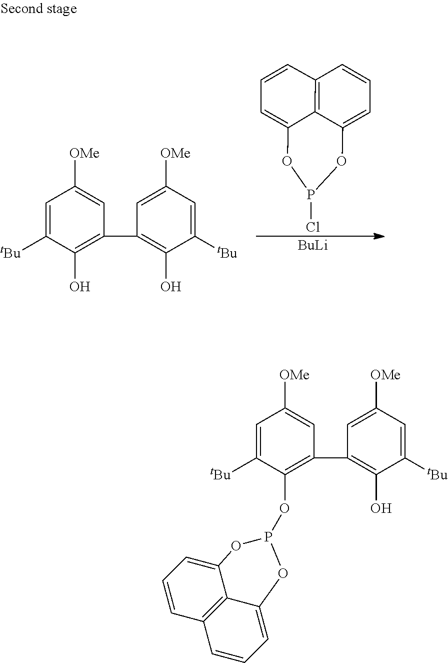

0.064 mol of biphenol was dried overnight by means of oil-pump vacuum. The next morning, the Schlenk flask was flooded with argon and the biphenol was dissolved in 200 ml of dried THF. Subsequently, the solution was cooled to −20° C. and 0.064 mol of a 1.6 M n-butyllithium solution in hexane was added dropwise. After addition was complete, the reaction solution was stirred at −20° C. for a further 30 min and then slowly heated to room temperature. Under an inert gas atmosphere, 0.065 mol of the chlorophosphite was dissolved in 100 ml of dried THF. Thereafter, the butyllithium-THF solution was slowly and steadily added at room temperature to the biphenol-THE solution. The reaction solution was stirred at room temperature overnight and the next morning was concentrated to dryness at 40° C. by means of oil-pump vacuum.

For purification, 700 ml of degassed n-heptane was added to the solid obtained and the mixture was heated to 70° C. and left to stir for 2 hours. The hot solution was filtered through Celite using a frit in order to separate off the precipitated salts. The solution was then cooled to 0° C. and stirred for four hours. Subsequently, the solid obtained was filtered off using a frit, washed twice with in each case 50 ml of cold heptane and dried by means of oil-pump vacuum.

Yield: 66%

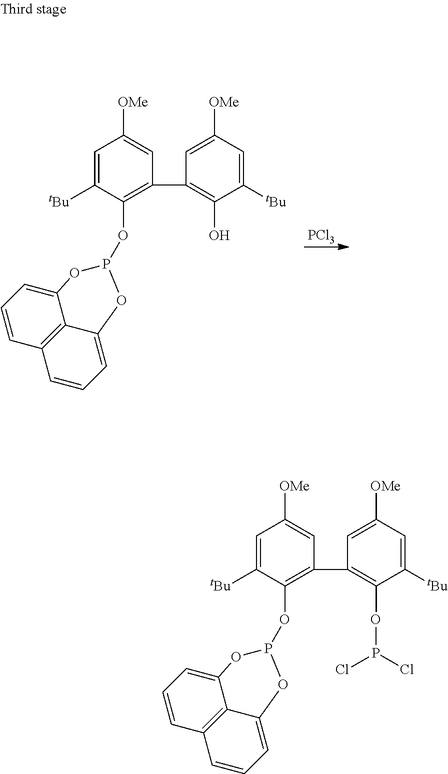

Under an inert gas atmosphere, 0.038 mol of organophosphite was dissolved in 200 ml of dried toluene and 13.5 ml (0.095 mol, 2.5 eq.) of triethylamine. In a Schlenk flask which had been repeatedly evacuated and filled with inert gas, 0.046 mol of phosphorus trichloride was dissolved in 100 ml of dried toluene and cooled to 0° C. The organophosphite-Et₃N solution was then slowly and steadily added dropwise at 0° C. to the PCl₃. The reaction solution was stirred at room temperature overnight. The ammonium hydrochloride formed was filtered off using a frit and washed twice with in each case 50 ml of dried toluene. The filtrate obtained was subsequently concentrated to dryness at 40° C. by means of oil-pump vacuum.

Yield: 91%

### Synthesis (1)

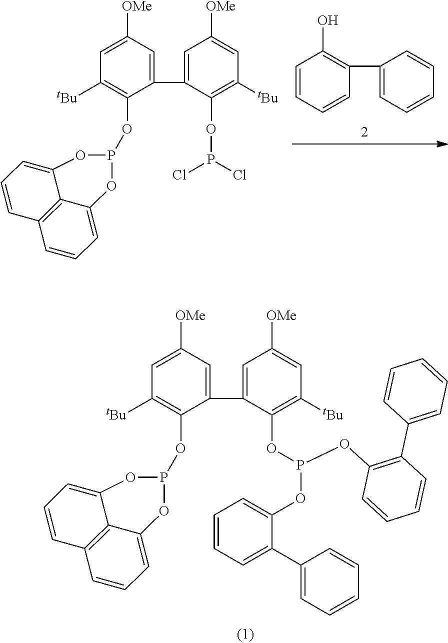

Under an inert gas atmosphere, 2.6 mmol of organodichlorophosphite was weighed out and suspended in 30 ml of dried toluene. 6.6 mmol of the phenol derivative was weighed out in a Schlenk flask and repeated evacuation and filling with inert gas was briefly performed by means of oil-pump vacuum. Subsequently, the Schlenk flask was flooded with argon and the phenol derivative was dissolved in 20 ml of dried toluene and 13.1 mmol of degassed triethylamine was added. Subsequently, the phenol solution was slowly and steadily added at room temperature to the chlorophosphite suspension. The reaction solution was stirred at room temperature overnight. The ammonium hydrochloride formed was filtered off using a frit and washed twice with in each case 10 ml of dried toluene. The filtrate obtained was subsequently concentrated to dryness at 40° C. by means of oil-pump vacuum. The dried filtrate was purified by means of column chromatography.

Yield: 78%

Compounds (2) to (6), (IIa) and (7) were prepared analogously to Compound (1).

Compounds (3) to (6), (IIa) and (7) are non-inventive comparative compounds.

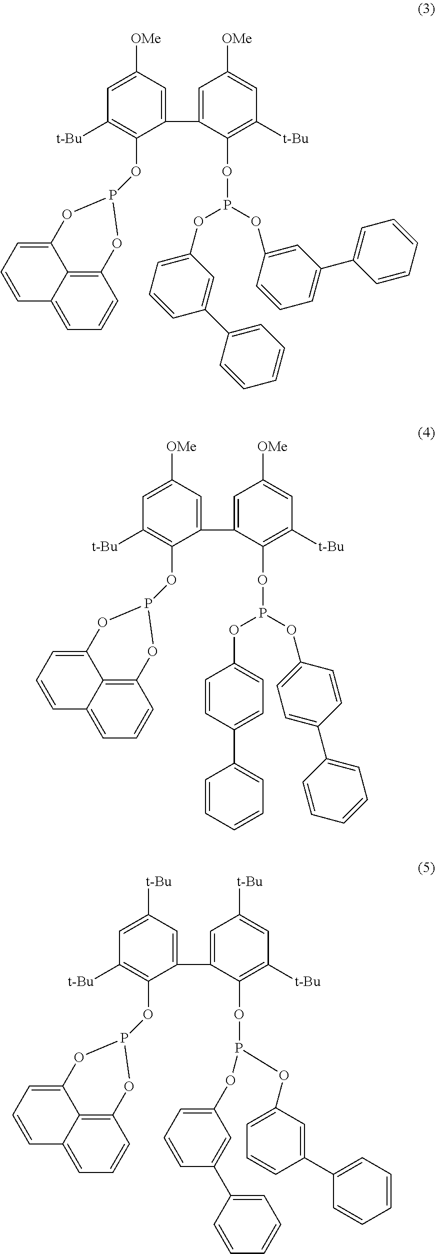 [SMILES: COc1cc(-c2cc(OC)cc(C(C)(C)C)c2OP2Oc3cccc4cccc(c34)O2)c(OP(Oc2cccc(-c3ccccc3)c2)Oc2cccc(-c3ccccc3)c2)c(C(C)(C)C)c1 ; COc1cc(-c2cc(OC)cc(C(C)(C)C)c2OP2Oc3cccc4cccc(c34)O2)c(OP(Oc2ccc(-c3ccccc3)cc2)Oc2ccc(-c3ccccc3)cc2)c(C(C)(C)C)c1 ; CC(C)(C)c1cc(-c2cc(C(C)(C)C)cc(C(C)(C)C)c2OP2Oc3cccc4cccc(c34)O2)c(OP(Oc2cccc(-c3ccccc3)c2)Oc2cccc(-c3ccccc3)c2)c(C(C)(C)C)c1]

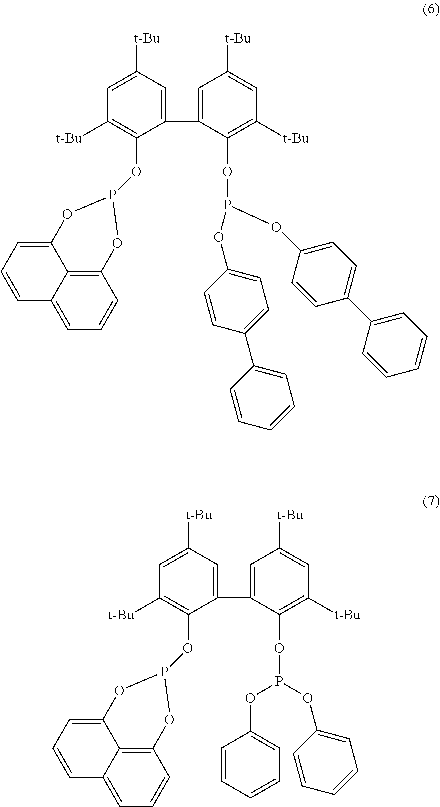 [SMILES: CC(C)(C)c1cc(-c2cc(C(C)(C)C)cc(C(C)(C)C)c2OP2Oc3cccc4cccc(c34)O2)c(OP(Oc2ccc(-c3ccccc3)cc2)Oc2ccc(-c3ccccc3)cc2)c(C(C)(C)C)c1 ; CC(C)(C)c1cc(-c2cc(C(C)(C)C)cc(C(C)(C)C)c2OP2Oc3cccc4cccc(c34)O2)c(OP(Oc2ccccc2)Oc2ccccc2)c(C(C)(C)C)c1]

### Catalysis Experiments

An argon atmosphere is employed. Reaction vessels have been dried beforehand at high temperature (80° C.) and under oil-pump vacuum. Liquid substances were degassed by bubbling in argon for at least 15 minutes. The hydroformylation was carried out in a 0.5 l autoclave from Berghof Products+Instruments GmbH, equipped with a constant-pressure apparatus. The reactor is heated by means of oil bath from IKA. The reactor serves for gas exchange and as a temperature control unit. Five glass vials (20 ml), filled with catalyst solution and magnetic stirrer bars and crimped under argon, were placed inside this reactor such that gas exchange between vials and reactor space is possible. The temperature of the glass vials was controlled by thermal oil present in the reactor. Reaction temperatures specified were measured inside the glass vials. The substrate used was n-octene (Oxeno GmbH, octene isomer mixture of 1-octene: 3%; cis+trans-2-octene: 49%; cis+trans-3-octene: 29%; cis+trans-4-octene: 16%; structurally isomeric octenes: 3%).

For an experiment run, a stock solution was prepared in advance under an argon atmosphere. For this purpose, 0.0127 g of Rh(acac)(CO)₂ and the corresponding amount of phosphite compound (MV Lig:Rh=5:1) were weighed out and made up with 48.0 ml of toluene. Approx. 8 ml of this solution was distributed to each of the vials and the exact amount was weighed. The vials were placed in the reactor and the latter was closed. Purging was performed three times with argon and three times with synthesis gas (Linde; H₂ (99.999%):CO (99.997%)=1:1). On completion of pressure testing, the autoclave was heated to the desired temperature of 120° C. at an overall pressure of 10 bar with stirring (900 rpm). After reaching the reaction temperature, the synthesis gas pressure was increased to 20 bar and 2 ml of the substrate in each case was metered in by means of HPLC pump to start the reaction. This results in an Rh concentration of 100 ppm. 1 h after the start of the reaction at constant pressure, a sample was taken from each vial and analysed undiluted by gas chromatography: HP 6890, Petrocol® DH 150, 150 m×0.25 mm×1 μm. Residual olefin and aldehyde were quantitatively determined against the solvent toluene as internal standard. The results listed in the table below are the average over one experiment run.

### Results of the Catalysis Experiments

[Rh]: 100 ppm, p: 20 bar, T: 120° C.; t: 1 h

The n-octene mixture used consisted of the C8 isomers: 1-octene, cis-2-octene, trans-2-octene, cis-3-octene, trans-3-octene, cis-4-octene and trans-4-octene.

TABLE 1
(R² = —O—Me):

| Ligand | n-Selectivity [%] |
|---|---|
| (1)* | 91 |
| (3) | 87 |
| (4) | 87 |
| (IIa) | 87 |

*inventive exemplary embodiment

TABLE 2
(R² = —^{t}Bu):

| Ligand | n-Selectivity [%] |
|---|---|
| (2)* | 91 |
| (5) | 83 |
| (6) | 81 |
| (7) | 84 |

*inventive exemplary embodiment

The experiments carried out demonstrate that the stated object is achieved by a compound according to the invention.

1. Compound of formula (I): 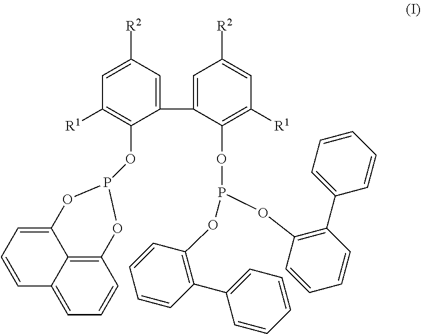
wherein R¹, R² are selected from: —H, —(C₁-C₆)-alkyl, —O—(C₁-C₆)-alkyl.

2. Compound according to claim 1, wherein R¹, R² are selected from: —(C₁-C₆)-alkyl, —O—(C₁-C₆)-alkyl.

3. Compound according to claim 1, wherein R¹ is —(C₁-C₆)-alkyl.

4. Compound according to claim 1, wherein R¹ is -^{t}Bu.

5. Compound according to claim 1, wherein R² is selected from: —(C₁-C₆)-alkyl, —O—(C₁-C₆)-alkyl.

6. Compound according to claim 1, wherein R² is -^{t}Bu or —O—CH₃.

7. Compound according to claim 1, wherein R² is -^{t}Bu.

8. Compound according to claim 1, wherein R² is —O—CH₃.

9. Compound according to claim 1, wherein the compound has the structure (1) or (2):

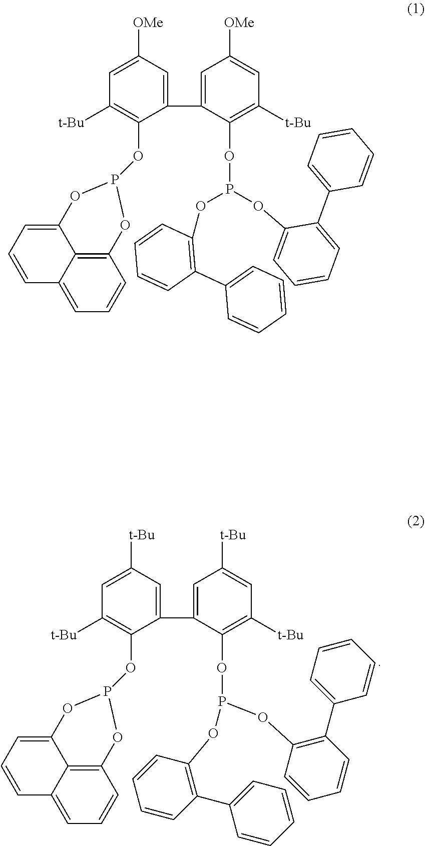 [SMILES: COc1cc(-c2cc(OC)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1 ; CC(C)(C)c1cc(-c2cc(C(C)(C)C)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

10. Compound according to claim 1, wherein the compound has the structure (1):

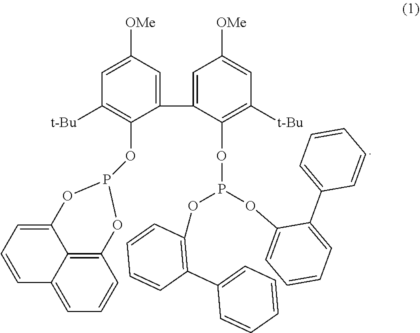 [SMILES: COc1cc(-c2cc(OC)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

11. Compound according to claim 1, wherein the compound has the structure (2):

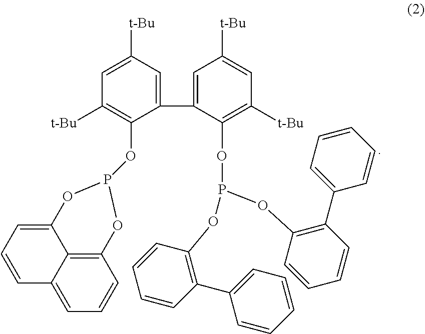 [SMILES: CC(C)(C)c1cc(-c2cc(C(C)(C)C)cc(C(C)(C)C)c2OP2Oc3cccc4cccc(c34)O2)c(OP(Oc2ccccc2-c2ccccc2)Oc2ccccc2-c2ccccc2)c(C(C)(C)C)c1]

12. Process comprising the process steps of:
a) initially charging an olefin;
b) adding a compound according to claim 1;
c) adding a substance comprising Rh;
d) feeding in H₂ and CO;
e) heating the reaction mixture from a) to d), with conversion of the olefin to an aldehyde.

13. Process according to claim 12, wherein the substance comprising Rh is selected from: Rh(acac)(CO)₂, Rh(acac)(cod) (Umicore, acac=acetylacetonate anion; cod=1,5-cyclooctadiene), Rh₄CO₁₂.

14. Process according to claim 12, wherein the substance comprising Rh is Rh(acac)(CO)₂.
